(19) United States
(12) Patent Application Publication
(10) Pub. No.: US 2009/0215678 A1
(43) Pub. Date: Aug. 27, 2009

(54) Preventing Vertical Endoparasite Infections

(75) Inventors: Thomas Bach, Wuppertal (DE); Janina Tanzler, Nieder-Olm (DE)

Correspondence Address: BAYER HEALTHCARE LLC, P.O.BOX 390, SHAWNEE MISSION, KS, 66201, US

(73) Assignee: BAYER HEALTHCARE AG, D-51368 LEVERKUSEN (DE)

(21) Appl. No.: 11/719,379

(22) PCT Filed: Nov. 3, 2005
(86) PCT No.: PCT/EP05/11748

§ 371 Date: May 15, 2007

(30) Foreign Application Priority Data
Nov. 16, 2004 (DE) 10 2004 055 316.5

(51) Int. Cl. A61K 38/12; C07K 5/12; A61P 33/00

(52) U.S. Cl. 514/11; 530/317

(57) ABSTRACT

The present invention relates to the use of endoparasiticidal depsipeptides for producing pharmaceuticals for preventing vertical infection with endoparasites.

The present invention relates to the use of endoparasiticidal depsipeptides for producing pharmaceuticals for preventing vertical infection with endoparasites.

The cyclic depsipeptide PF1022, and its effect against endoparasites, are disclosed in EP-A 382 173. Patent applications EP-A 626 376; EP-A 626 375 and EP-A 644 883 relate to other cyclic depsipeptides and their endoparasiticidal effect.

Endoparasiticidal compositions comprising praziquantel or epsiprantel and cyclic depsipeptides are described in EP 662 326.

It is known that, after having been administered orally, parenterally or transdermally, these compositions have an effect against endoparasites in the animal concerned. However, it has not previously been known that these active compounds are also able to prevent vertical infections in the progeny of the animals.

The invention therefore relates to:

The use of endoparasiticidal depsipeptides for producing pharmaceuticals for preventing vertical infection with endoparasites.

Depsipeptides are similar to peptides and differ from the latter in that one or more α-amino acid building blocks has/have been replaced with α-hydroxycarboxylic acid building blocks. According to the invention, preference is given to using cyclic depsipeptides having from 18 to 24 ring atoms, in particular having 24 ring atoms.

The depsipeptides having 18 ring atoms include compounds of the general formula (I);

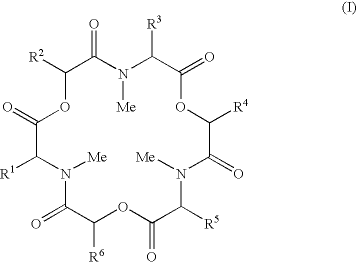

in which
R¹, R³ and R⁵ are, independently of each other, hydrogen, straight-chain or branched alkyl having up to 8 carbon atoms, hydroxyalkyl, alkanoyloxyalkyl, alkoxyalkyl, aryloxyalkyl, mercaptoalkyl, alkylthioalkyl, alkylsulfinylalkyl, alkylsulfonylalkyl, carboxyalkyl, alkoxycarbonylalkyl, arylalkoxycarbonylalkyl, carbamoylalkyl, aminoalkyl, alkylaminoalkyl, dialkylaminoalkyl, guanidinoalkyl, which can be optionally substituted by one or two benzyloxycarbonyl radicals or by one, two, three or four alkyl radicals, alkoxycarbonylaminoalkyl, 9-fluorenylmethoxycarbonyl(Fmoc)aminoalkyl, alkenyl, cycloalkyl, cycloalkylalkyl and optionally substituted arylalkyl, with substituents which may be mentioned being halogen, hydroxyl, alkyl and alkoxy,
R², R⁴ and R⁶ are, independently of each other, hydrogen, straight-chain or branched alkyl having up to 8 carbon atoms, hydroxyalkyl, mercaptoalkyl, alkanoyloxyalkyl, alkoxyalkyl, aryloxyalkyl, alkylthioalkyl, alkylsulfinylalkyl, alkylsulfonylalkyl, carboxyalkyl, alkoxycarbonylalkyl, arylalkoxycarbonylalkyl, carbamoylalkyl, aminoalkyl, alkylaminoalkyl, dialkylaminoalkyl, alkoxycarbonylaminoalkyl, alkenyl, cycloalkyl, cycloalkylalkyl and optionally substituted aryl or arylalkyl, with substituents which may be mentioned being halogen, hydroxyl, alkyl and alkoxy,
   and the optical isomers and racemates thereof.

Preference is given to compounds of formula (I)

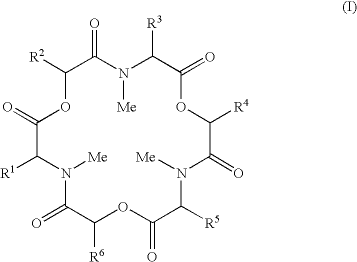

in which
R¹, R³ and R⁵ are, independently of each other, straight-chain or branched C₁-C₈-alkyl, in particular methyl, ethyl, propyl, isopropyl, butyl, isobutyl, sec-butyl, tertbutyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, mercapto-C₁-C₆-alkyl, in particular mercaptomethyl, C₁-C₄-alkylthio-C₁-C₆-alkyl, in particular methylthioethyl, C₁-C₄-alkylsulfinyl-C₁-C₆-alkyl, in particular methylsulfinylethyl, C₁-C₄-alkylsulfonyl-C₁-C₆-alkyl, in particular methylsulfonylethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, carbamoyl-C₁-C₆-alkyl, in particular carbamoylmethyl, carbamoylethyl, amino-C₁-C₆-alkyl, in particular aminopropyl, aminobutyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, guanido-C₁-C₆-alkyl, in particular guanidopropyl, C₁-C₄-alkoxycarbonylamino-C₁-C₆-alkyl, in particular tert-butoxycarbonylaminopropyl, tert-butoxycarbonylaminobutyl, 9-fluorenylmethoxycarbonyl(Fmoc)amino-C₁-C₆-alkyl, in particular 9-fluorenylmethoxycarbonyl(Fmoc)aminopropyl, 9-fluorenylmethoxycarbonyl(Fmoc)aminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl-C₁-C₄-alkyl, in particular phenylmethyl which can be optionally substituted by radicals from the group halogen, in particular fluorine, chlorine, bromine or iodine, hydroxyl, C₁-C₄-alkoxy, in particular methoxy or ethoxy, and C₁-C₄-alkyl, in particular methyl,
R², R⁴ and R⁶ are, independently of each other, straight-chain or branched C₁-C₈-alkyl, in particular methyl, ethyl, propyl, isopropyl, butyl, isobutyl, sec-butyl, tertbutyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, mercapto-C₁-C₆-alkyl, in particular mercaptomethyl, C₁-C₄-alkylthio-C₁-C₆-alkyl, in particular methylthioethyl, C₁-C₄-alkylsulfinyl-C₁-C₆-alkyl, in particular methylsulfinylethyl, C₁-C₄-alkylsulfonyl-C₁-C₆-alkyl, in particular methylsulfonylethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, carbamoyl-C₁-C₆-alkyl, in particular carbamoylmethyl, carbamoylethyl, amino-C₁-C₆-alkyl, in particular aminopropyl, aminobutyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl, phenyl-C₁-C₄-alkyl, in particular phenylmethyl which can be optionally substituted by radicals from the group halogen, in particular fluorine, chlorine, bromine or iodine, hydroxyl, C₁-C₄-alkoxy, in particular methoxy or ethoxy, and C₁-C₄-alkyl, in particular methyl, and the optical isomers and racemates thereof.

Particular preference is given to compounds of formula (I),

in which
R¹, R³ and R⁵ are, independently of each other, straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, C₁-C₄-alkoxycarbonylamino-C₁-C₆-alkyl, in particular tert-butoxycarbonylaminopropyl, tert-butoxycarbonylaminobutyl, C₂-C₈-alkenyl, in particular vinyl, allyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl-C₁-C₄-alkyl, in particular phenylmethyl which can be optionally substituted by one or more identical or different radicals from those specified above,
R², R⁴ and R⁶ are, independently of each other, straight-chain or branched C₁-C₈-alkyl, in particular methyl, ethyl, propyl, isopropyl, butyl, isobutyl, sec-butyl, tertbutyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, aryl-C₁-C₄-alkyloxy-C₁-C₆-alkyl, in particular benzyloxymethyl, 1-benzyloxyethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl, phenyl-C₁-C₄-alkyl, in particular phenylmethyl which can be optionally substituted by one or more identical or different radicals from those specified above,
   and the optical isomers and racemates thereof.

Very particular preference is given to compounds of formula (I),

in which
R¹, R³ and R⁵ are, independently of each other, straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, C₂-C₈-alkenyl, in particular allyl, C₃-C₇-cycloalkyl-C₁-C₄-alkyl, in particular cyclohexylmethyl, phenyl-C₁-C₄-alkyl, in particular phenylmethyl,
R², R⁴ and R⁶ are, independently of each other, straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, C₂-C₈-alkenyl, in particular vinyl, allyl, C₃-C₇-cycloalkyl-C₁-C₄-alkyl, in particular cyclohexylmethyl, phenyl-C₁-C₄-alkyl, in particular phenylmethyl which can be optionally substituted by one or more identical or different radicals from those specified above,
   and the optical isomers and racemates thereof.

In detail, mention may be made of the following compounds of the general formula (I)

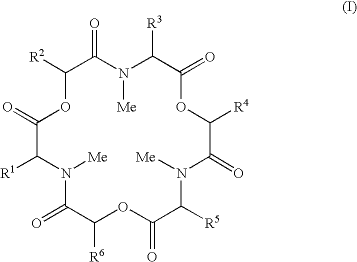

in which the radicals R¹ to R⁶ have the following meanings:

| R¹ | R² | R³ | R⁴ | R⁵ | R⁶ |
|---|---|---|---|---|---|
| —CHMeCH₂Me | -cyclohexyl | —CHMeCH₂Me | -Me | —CHMeCH₂Me | -Me |
| —CHMeCH₂Me | -cyclohexyl | —CHMeCH₂Me | -Me | —CHMeCH₂Me | -cyclohexyl |
| —CHMeCH₂Me | —CH₂-Phe | —CHMeCH₂Me | -Me | —CHMeCH₂Me | -Me |
| —CHMeCH₂Me | —CH₂-Phe | —CHMeCH₂Me | -Me | —CHMeCH₂Me | —CH₂-Phe |
| —CHMeCH₂Me | —(CH₂)₃-Me | —CHMeCH₂Me | -Me | —CHMeCH₂Me | —Me |
| —CHMeCH₂Me | —(CH₂)₃-Me | —CHMeCH₂Me | -Me | —CHMeCH₂Me | —(CH₂)₃-Me |
| —CHMe₂ | —CH₂-Phe | —CHMeCH₂Me | -Me | —CHMeCH₂Me | —Me |
| —CH₂-Phe | —CHMe₂ | —CH₂-Phe | —CHMe₂ | —CHMeCH₂Me | —CHMe₂ |
| —CH₂CHMe₂ | —CH₂-Phe | —CH₂CHMe₂ | -Me | —CH₂CHMe₂ | —CH₂-Phe |
| —(CH₂)₃-Me | -Me | —CHMeCH₂Me | -Me | —CHMeCH₂Me | -Me |
| —CHMe₂ | -Me | —CHMe₂ | -Me | —CHMe₂ | -Me |
| —CH₂-Me | -Me | —CH₂-Me | -Me | —CH₂-Me | -Me |
| —(CH₂)₂-Me | -Me | —(CH₂)₂-Me | -Me | —(CH₂)₂-Me | -Me |
| —(CH₂)₃-Me | -Me | —(CH₂)₃-Me | -Me | —(CH₂)₃-Me | -Me |
| —CH₂—CH═CH₂ | -Me | —CH₂—CH═CH₂ | -Me | —(CH₂)—CH═CH₂ | -Me |
| —CHMeCH₂Me | -Me | —CHMeCH₂Me | -Me | —CHMeCH₂Me | —CH₂-Me |
| —CHMeCH₂Me | -Me | —CHMeCH₂Me | -Me | —CHMeCH₂Me | —(CH₂)₂-Me |
| —CHMeCH₂Me | -Me | —CHMeCH₂Me | -Me | —CHMeCH₂Me | —(CH₂)₃-Me |
| —CHMeCH₂Me | -Me | —CHMeCH₂Me | -Me | —CH₂Me | -Me |
| —CHMeCH₂Me | -Me | —CHMeCH₂Me | -Me | —(CH₂)₂-Me | -Me |
| -cyclohexyl | -Me | -cyclohexyl | -Me | -cyclohexyl | -Me |
| —CH₂CHMe₂ | -cyclohexyl | —CH₂CHMe₂ | -Me | —CH₂CHMe₂ | -cyclohexyl |
| —CH₂CHMe₂ | -cyclohexyl | —CH₂CHMe₂ | -Me | —CH₂CHMe₂ | -Me |
| —CHMeCH₂Me | —CHMe₂ | —CHMeCH₂Me | —CHMe₂ | —CHMeCH₂Me | -Me |
| —CH₂-Phe | -Me | —CH₂-Phe | -Me | —CH₂-Phe | -Me |
| -cyclohexyl | -Me | -cyclohexyl | -Me | -cyclohexyl | -Me |
| —CHMe₂ | —CHMe₂ | —CHMe | -Me | —CHMe₂ | -Me |
| —CHMe₂ | —CHMe₂ | —CHMe₂ | —CHMe₂ | —CHMe₂ | -Me |
| —CH₂-Me | —CHMe₂ | —CH₂Me | -Me | —CH₂-Me | -Me |
| —CH₂-Me | —CHMe₂ | —CHMe₂ | —CHMe₂ | —CH₂-Me | -Me |
| —(CH₂)₂-Me | —CHMe₂ | —(CH₂)₂-Me | -Me | —(CH₂)₂-Me | -Me |
| —(CH₂)₂-Me | —CHMe₂ | —(CH₂)₂-Me | —CHMe₂ | —(CH₂)₂-Me | -Me |
| —(CH₂)₃-Me | —CHMe₂ | —(CH₂)₃-Me | -Me | —(CH₂)₃-Me | -Me |
| —(CH₂)₃-Me | —CHMe₂ | —(CH₂)₃-Me | —CHMe₂ | —(CH₂)₃-Me | -Me |
| —CH₂—CH═CH₂ | —CHMe₂ | —CH₂—CH═CH₂ | -Me | —CH₂—CH═CH₂ | -Me |
| —CH₂—CH═CH₂ | —CHMe₂ | —CH₂—CH═CH₂ | —CHMe₂ | —CH₂—CH═CH₂ | -Me |
| -Me | -Me | —CHMeCH₂Me | -Me | —CH₂-Me | -Me |
| -Me | -Me | —CHMeCH₂Me | -Me | —(CH₂)₃-Me | -Me |

Me = methyl; Phe = phenyl

A further depsipeptide which may be mentioned is the compound PF 1022, which is disclosed in EP-OS 382 173 and which has the following formula (IIa):

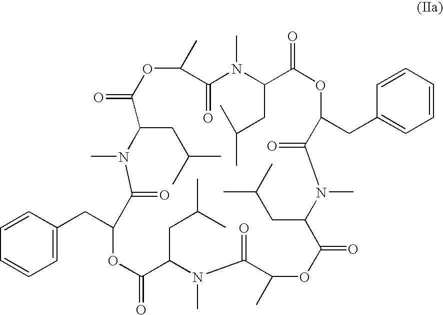

Additional depsipeptides which may be mentioned are the compounds which are disclosed in PCT application WO 93/19053.

Particular mention may be made of the compounds which are disclosed in WO 93/19053 and which have the following formula (IIb):

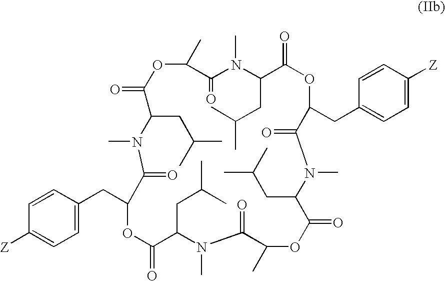

in which
Z is N-morpholinyl, amino, mono- or dimethylamino.

Mention may also be made of compounds which have the following formula (IIc):

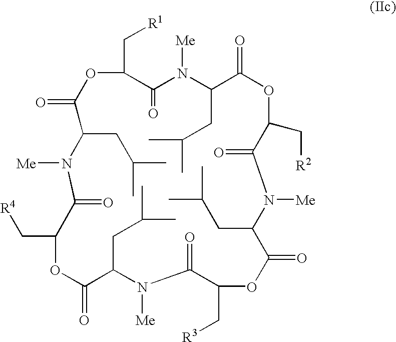

in which
R¹, R², R³ and R⁴ are, independently of each other, hydrogen, C₁-C₁₀-alkyl or aryl, in particular phenyl, which are optionally substituted by hydroxyl, C₁-C₁₀-alkoxy or halogen.

The compounds of the general formula (I) are known and can be obtained using the methods described in EP-A-382 173, DE-A 4 317 432, DE-A 4 317 457, DE-A 4317 458, EP-A-634 408, EP-A-718 293, EP-A-872 481, EP-A-685 469, EP-A-626 375, EP-A-664 297, EP-A-669 343, EP-A-787 141, EP-A-865 498 and EP-A-903 347.

The cyclic depsipeptides having 24 ring atoms also include compounds of the general formula (IId)

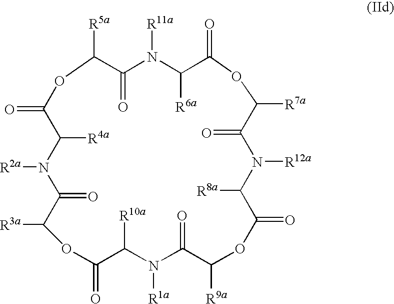

in which
R^{1a}, R^{2a}, R^{11a} and R^{12a} are, independently of each other, C₁₋₈-alkyl, C₁₋₈-halogenoalkyl, C₃₋₆-cycloalkyl, aralkyl, or aryl,
R^{3a}, R^{5a}, R^{7a} and R^{9a} are, independently of each other, hydrogen or straight-chain or branched C₁₋₈-alkyl which can be optionally substituted by hydroxyl, C₁₋₄-alkoxy, carboxyl,

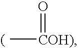

carboxamide,

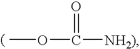

imidazolyl, indolyl, guanidino, —SH or C₁₋₄-alkylthio, and are, additionally, aryl or aralkyl which can be substituted by halogen, hydroxyl, C₁₋₄-alkyl or C₁₋₄-alkoxy,
R^{4a}, R^{6a}, R^{8a} and R^{10a} are, independently of each other, hydrogen, straight-chain C₁₋₅-alkyl, C₂₋₆-alkenyl or C₃₋₇-cycloalkyl which can be substituted by hydroxyl, C₁₋₄-alkoxy, carboxyl, carboxamide, imidazolyl, indolyl, guanidino, SH or C₁₋₄-alkylthio, and are also aryl or aralkyl which can be substituted by halogen, hydroxyl, C₁₋₄-alkyl or C₁₋₄-alkoxy,
   and the optical isomers and racemates thereof.

Preference is given to using compounds of the formula (IId) in which
R^{1a}, R^{2a}, R^{11a} and R^{12a} are, independently of each other, methyl, ethyl, propyl, isopropyl, n-, s- or t-butyl or phenyl which is optionally substituted by halogen, C₁₋₄-alkyl, OH or C₁₋₄-alkoxy, and are also benzyl or phenylethyl which can be optionally substituted by the radicals mentioned in the case of phenyl; and
R^{3a} to R^{10a} have the abovementioned meaning.

Particular preference is given to compounds of formula (IId) in which
R^{1a}, R^{2a}, R^{11a} and R^{12a} are, independently of each other, methyl, ethyl, propyl, isopropyl or n-, s- or t-butyl,
R^{3a}, R^{5a}, R^{7a} and R^{9a} are hydrogen or straight-chain or branched C₁₋₈-alkyl, in particular methyl, ethyl, propyl, i-propyl or n-, s- or t-butyl, which radical can be optionally substituted by C₁₋₄-alkoxy, in particular methoxy or ethoxy, imidazolyl, indolyl or C₁₋₄-alkylthio, in particular methylthio or ethylthio, and are also phenyl, benzyl or phenethyl which can be optionally substituted by halogen, in particular chlorine.
R^{4a}, R^{6a}, R^{8a} and R^{10a} are, independently of each other, hydrogen, methyl, ethyl, n-propyl, n-butyl, vinyl or cyclohexyl, which radical can be optionally substituted by methoxy, ethoxy, imidazolyl, indolyl, methylthio or ethylthio, and are also isopropyl or s-butyl and, in addition, phenyl, benzyl or phenylethyl which are optionally substituted by halogen.

The compounds of the formula (IId) can also be obtained using the methods described in EP-A-382 173, DE-A 4 317 432, DE-A 4 317 457, DE-A 4 317 458, EP-A-634 408, EP-A-718 293, EP-A-872 481, EP-A-685 469, EP-A-626 375, EP-A-664 297, EP-A-669 343, EP-A-787 141, EP-A-865 498 and EP-A-903 347.

Depsipeptides which are very particularly preferred in accordance with the invention are PF 1022 A (see formula (IIa)) and emodepside (PF 1022-221, compound of the formula (IIb) in which both the Z radicals are the morpholinyl radical). The INN emodepside is the compound having the systematic name: cyclo[(R)-lactoyl-N-methyl-L-leucyl-(R)-3-(p-morpholinophenyl)lactoyl-N-methyl-L-leucyl-(R)-lactoyl-N-methyl-L-leucyl-(R)-3-(p-morpholinophenyl)lactoyl-N-methyl-L-leucyl.

Depending on their structure, active compounds may be present in stereoisomeric forms or as stereoisomeric mixtures, e.g. as enantiomers or racemates. Both the stereoisomeric mixtures and the pure stereoisomers can be used in accordance with the invention.

The following can also be used, where appropriate: salts of the active compounds with pharmaceutically acceptable acids or bases, and also solvates, in particular hydrates, of the active compounds or their salts.

According to the invention, the active compounds can be used for controlling pathogenic endoparasites which are found in humans and in animal husbandry and animal breeding, in the case of productive animals, breeding animals, zoo animals, laboratory animals, experimental animals and pet animals. In this connection, the compounds of the invention are effective against all, or individual, developmental stages of the pests as well as against resistant species and species which are normally susceptible. Control of the pathogenic endoparasites is intended to reduce disease, mortality and decreases in production (e.g. in the production of meat, milk, wool, hides, eggs, etc.) such that using the active compounds makes it possible to achieve an animal husbandry which is simpler and more economic. In a general manner, the pathogenic endoparasites include cestodes, trematodes, nematodes, acantocephala. Preference is given to using depsipeptides to control infections due to nematodes; the following endoparasites may be mentioned individually:

From the order of the Enoplida, e.g.: Trichuris spp., Capillaria spp., Trichomosoides spp. and Trichinella spp.

From the order of the Rhabditia, e.g.: Micronema spp. and Strongyloides spp.

From the order of the Strongylida, e.g.: Stronylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp. and Ollulanus spp.

From the order of the Oxyurida, e.g.: Oxyuris spp., Enterobius spp., Passalurus spp., Syphacia spp., Aspiculuris spp. and Heterakis spp.

From the order of the Ascaridia, e.g.: Ascaris spp., Toxascaris spp., Toxocara spp., Parascaris spp., Anisakis spp., Ascaridia spp. and Baylisascaris spp.

From the order of the Spirurida, e.g.: Gnathostoma spp., Physaloptera spp., Thelazia spp., Gongylonema spp., Habronema spp., Parabronema spp., Draschia spp. and Dracunculus spp.

From the order of the Filariida, e.g.: Stephanofilaria spp., Parafilaria spp., Setaria spp., Loa spp., Dirofilaria spp., Litomosoides spp., Brugia spp., Wuchereria spp. and Onchocerca spp.

From the order of the Gigantorhynchida, e.g.: Filicollis spp., Moniliformis spp., Macracanthorhynchus spp. and Prosthenorchis spp.

Preference is given, according to the invention, to using the active compounds against infections with helminths from the order of the Rhabditia, e.g., Strongyloides spp. and from the order of the Strongylida, e.g. Ancylostoma spp., and also against infections with the helminths which are mentioned above in connection with the order of the Ascaridia. Particular preference is given to using the active compounds against Toxocara spp., e.g. Toxocara canis.

The productive and breeding animals include mammals such as cattle, horses, sheep, pigs, goats, camels, water buffaloes, donkeys, rabbits, fallow deer, reindeer, and fur animals such as mink, chinchilla and raccoon.

Laboratory and experimental animals include mice, rats, guinea pigs, golden hamsters, dogs and cats.

The pet animals include dogs and cats.

The active compounds can be used either prophylactically or therapeutically. Very particular preference is given to using the active compounds in dogs.

When used in accordance with the invention, the depsipeptides proved to be well tolerated both with regard to the dams and with regard to the progeny.

Vertical infection means that transfer takes place from the dam to the progeny, with this transfer taking place, in particular, prenatally, i.e. in the maternal body, and/or galactogenically, that is by way of the milk. The prevention of prenatal infection is of particular importance.

“Preventing prenatal and galactogenic infection” does not only mean complete prevention of an infection; it also means incomplete prevention in which the risk of an infection is only reduced and/or the infection is attenuated. That which is to be aspired to is an efficacy of at least 90%.

The active compounds are used directly or in the form of suitable preparations, with the administration preferably being effected enterally, parenterally or dermally (transdermally) into the dam.

The active compounds are used enterally, for example, orally in the form of powders, suppositories, tablets, capsules, pastes, potions, granules, drenches, boli or medicated feed or drinking water. They are used dermally, for example, in the form of dipping, spraying, bathing, washing, pouring-on and spotting-on and powdering-in. They are used parenterally, for example, in the form of injection (intramuscular, subcutaneous, intravenous or intraperitoneal) or by means of implants.

Suitable preparations are:

solutions such as injection solutions, oral solutions, concentrates for oral administration after having been diluted, solutions for use on the skin or in body cavities, pour-on formulations and gels;
emulsions and suspensions for oral or dermal use and for injection; semisolid preparations;
formulations in which the active compound is worked into an ointment base or into an oil-in-water or water-in-oil emulsion base;
solid preparations such as powders, premixes or concentrates, granules, pellets, tablets, boli, capsules; aerosols and inhalates, and active compound-containing moulded bodies.

The injection solutions are administered intravenously, intramuscularly and subcutaneously.

The injection solutions are prepared by dissolving the active compound in a suitable solvent and, where appropriate, adding additives such as solubilizers, acids, bases, buffer salts, antioxidants and preservatives. The solutions are sterilized by filtration and aliquotted out.

Solvents which may be mentioned are: physiologically tolerated solvents such as water, alcohols, such as ethanol, butanol, benzyl alcohol and glycerol, hydrocarbons, propylene glycol, polyethylene glycols and N-methylpyrrolidone and mixtures thereof.

Where appropriate, the active compounds can also be dissolved in physiologically tolerated vegetable or synthetic oils which are suitable for injection.

Solubilizers which may be mentioned are: solvents which promote the dissolution of the active compound in the main solvent or prevent it from being precipitated out. Examples are polyvinylpyrrolidone, polyethoxylated castor oil and polyethoxylated sorbitan esters.

Preservatives are: benzyl alcohol, trichlorobutanol, p-hydroxybenzoic acid esters and n-butanol.

Oral solutions are used directly. Concentrates are used orally after having been previously diluted down to the use concentration. Oral solutions and concentrates are prepared as described above in connection with the injection solutions, with it being possible to dispense with the need to operate under sterile conditions.

Solutions for use on the skin are dripped on, painted on, rubbed in, sprinkled on or sprayed on or applied by immersion (dipping, bathing or washing). These solutions are prepared as described above in the case of the injection solutions.

It may be advantageous to add thickeners during the preparation.

Thickeners are: inorganic thickeners, such as bentonites, colloidal silicic acid and aluminium monostearate, and organic thickeners, such as cellulose derivatives, polyvinyl alcohols and their copolymers, acrylates and methacrylates.

Gels are applied to the skin or painted on or introduced into body cavities. Gels are prepared by treating solutions, which have been prepared as described in the case of the injection solutions, with sufficient thickener to form a clear mass having an ointment-like consistency. The thickeners employed are those which are mentioned above.

Pour-on formulations are poured or sprinkled onto defined areas of the skin, with the active compound either penetrating the skin and acting systemically or being dispersed over the body surface.

Pour-on formulations are prepared by dissolving, suspending or emulsifying the active compound in suitable, skin-compatible solvents or solvent mixtures. Where appropriate, further auxiliary substances, such as colorants, absorption-promoting substances, antioxidants, light-stability agents and adhesives, are added.

Solvents which may be mentioned are: water, alkanols, glycols, polyethylene glycols, polypropylene glycols, glycerol, aromatic alcohols such as benzyl alcohol, phenylethanol and phenoxyethanol, esters, such as ethyl acetate, butyl acetate and benzylbenzoate, ethers, such as alkylene glycol alkyl ethers, such as dipropylene glycol monomethyl ether and diethylene glycol monobutyl ether, ketones, such as acetone and methyl ethyl ketone, aromatic and/or aliphatic hydrocarbons, vegetable or synthetic oils, DMF, dimethylacetamide, N-methylpyrrolidone and 2-dimethyl-4-oxymethylene-1,3-dioxolane.

Colorants are all colorants which are approved for use on animals and which can be dissolved or suspended.

Examples of absorption-promoting substances are DMSO, spreading oils such as isopropyl myristate, dipropylene glycol pelargonate, silicone oils, fatty acid esters, triglycerides and fatty alcohols.

Antioxidants are sulphites or metabisulphites such as potassium metabisulphite, ascorbic acid, butylhydroxytoluene, butylhydroxyanisole and tocopherol.

Examples of light-stability agents are substances from the benzophenone class or novantisolic acid.

Examples of adhesives are cellulose derivatives, starch derivatives, polyacrylates and natural polymers such as alginates and gelatin.

Emulsions can be used orally, dermally or as an injection.

Emulsions are either of the water-in-oil type or of the oil-in-water type.

They are prepared by dissolving the active compound either in the hydrophobic phase or in the hydrophilic phase and homogenizing the latter with the solvent of the other phase using suitable emulsifiers and, where appropriate, further auxiliary substances such as colorants, absorption-promoting substances, preservatives, antioxidants, light-stability agents and viscosity-increasing substances.

Hydrophobic phases (oils) which may be mentioned are: paraffin oils, silicone oils, natural plant oils, such as sesame oil, almond oil and castor oil, synthetic triglycerides such as caprylic/capric acid diglyceride, triglyceride mixtures containing plant fatty acids of C₈₋₁₂ chain length or other specially selected natural fatty acids, partial glyceride mixtures of saturated and unsaturated, where appropriate also hydroxyl group-containing, fatty acids, and mono- and diglycerides of the C₈/C₁₀ fatty acids.

Fatty acid esters such as ethyl stearate, di-n-butyryl adipate, hexyl laurate and dipropylene glycol pelargonate, esters of a branched fatty acid of medium chain length with saturated fatty alcohols of C₁₆-C₁₈ chain length, isopropyl myristate, isopropyl palmitate, caprylic/capric acid esters of saturated fatty alcohols of C₁₂-C₁₈ chain length, isopropyl stearate, oleyl oleate, decyl oleate, ethyl oleate, ethyl lactate, waxy fatty esters such as artificial duck tail gland fat, dibutyl phthalate, diisopropyl adipate, ester mixtures related to the latter, and others.

Fatty alcohols such as isotridecyl alcohol, 2-octyldodecanol, cetylstearyl alcohol and oleyl alcohol.

Fatty acids such as oleic acid and its mixtures.

Hydrophilic phases which may be mentioned are:

water, alcohols such as propylene glycol, glycerol, sorbitol and their mixtures.

Emulsifiers which may be mentioned are: nonionic surfactants, e.g. polyethoxylated castor oil, polyethoxylated sorbitan monooleate, sorbitan monostearate, glycerol monostearate, polyoxyethyl stearate and alkylphenol polyglycol ethers;

ampholytic surfactants such as di-Na—N-lauryl-β-iminodipropionate or lecithin;
anionic surfactants, such as Na lauryl sulphate, fatty alcohol ether sulphates, mono/dialkyl polyglycol ether orthophosphoric acid ester monoethanolamine salt;
cationic surfactants such as cetyltrimethylammonium chloride.

Further auxiliary substances which may be mentioned are: viscosity-increasing and emulsion-stabilizing substances such as carboxymethyl cellulose, methyl cellulose and other cellulose and starch derivatives, polyacrylates, alginates, gelatine, gum arabic, polyvinylpyrrolidone, polyvinyl alcohol, copolymers composed of methyl vinyl ether and maleic anhydride, polyethylene glycols, waxes and colloidal silicic acid, or mixtures of the listed substances.

Suspensions can be used orally, dermally or as an injection. They are prepared by suspending the active compound in a suspending liquid, where appropriate in the added presence of further auxiliary substances such as wetting agents, colorants, absorption-promoting substances, preservatives, antioxidants and light-stability agents.

Suspending liquids which may be mentioned are all homogeneous solvents and solvent mixtures.

Wetting agents (dispersing agents) which may be mentioned are the above-specified surfactants.

Other auxiliary substances which may be mentioned are those specified above.

Semisolid preparations can be administered orally or dermally. They only differ from the above-described suspensions and emulsions in having a higher viscosity.

In order to produce solid preparations, the active compound is mixed with suitable excipients, where appropriate in the added presence of auxiliary substances, and brought into the desired form.

Excipients which may be mentioned are all physiologically tolerated, solid inert substances. Inorganic and organic substances are used for this purpose. Examples of inorganic substances are sodium chloride, carbonates such as calcium carbonate and hydrogen carbonates, aluminium oxides, silicic acids, argillaceous earths, precipitated or colloidal silicon dioxide and phosphates.

Examples of organic substances are sugar, cellulose, and foodstuffs and feedstuffs such as powdered milk, animal meals, flours and coarse cereal meals, and starches.

Auxiliary substances are preservatives, antioxidants and colorants which have already been listed above.

Other suitable auxiliary substances are lubricants and glidants such as magnesium stearate, stearic acid, talc, bentonites, disintegration-promoting substances such as starch or crosslinked polyvinylpyrrolidone, binding agents such as starch, gelatin or linear polyvinylpyrrolidone and also dry binding agents such as microcrystalline cellulose.

Preferred administration forms are orally, e.g. by means of a suitable tablet, or transdermally, e.g. by means of a suitable spot-on formulation.

The compositions according to the invention can additionally comprise synergists or further active compounds, e.g. those which act against pathogenic endoparasites. Examples of these active compounds are L-2,3,5,6-tetrahydro-6-phenylimidazothiazole, benzimidazole carbamates such as febantel, and, in addition, pyrantel, epsiprantel or macrocyclic lactones such as avermectin, ivermectin or selamectin. Particular preference is given to praziquantel as a combination partner, in particular together with emodepside.

Praziquantel has been known for a long time as a compound which is active against endoparasites (cf., e.g., U.S. Pat. No. 4,001,411). The advantageous combination of depsipeptides with praziquantel or epsiprantel is described in EP-A-662 326, which document is hereby expressly incorporated by reference.

Combinations of depsipeptides with piperazines are described in EP-A-1 189 615, which document is hereby expressly incorporated by reference.

Compositions which can be administered transdermally and which comprise cyclic depsipeptides for controlling endoparasites are described in our German patent application having the file number 10358525.7, which application is likewise pending.

In general, ready-to-use preparations comprise the active compounds at concentrations of in each case 0.01-25% by weight, preferably of 0.1-20% by weight.

The cyclic depsipeptides are usually employed in quantities of from 0.1 to 8% by weight, preferably of from 1 to 6% by weight.

Praziquantel is customarily used in quantities of from 1 to 25% by weight, preferably of from 5 to 15% by weight, particularly preferably of from 6 to 14% by weight.

The compositions are prepared by mixing the components in the appropriate quantities in suitable appliances. The procedure is preferably such that the liquid components are mixed, after which the solid components are added and a homogeneous solution is prepared.

In general, it has proved to be advantageous, for achieving effective results, to administer quantities of the mixture according to the invention of from about 0.1 to about 20 mg of active compound per kg of body weight per day. From 0.5 to 10 mg of active compound per kg of body weight is preferred.

The following examples explain the invention without limiting it.

### BIOLOGICAL EXAMPLE

The investigations were carried out on eighteen beagle bitches which had been experimentally infected with Toxocara canis. Six of these animals served as the untreated control group. Two different treatment schemes were used. In treatment group 1 (T1), six bitches were treated daily, from the 42nd day of pregnancy onwards until the birth of the pups, with in each case 1 mg of emodepside/kg of body weight, given orally as a tablet formulation; the six bitches in treatment group 2 (T2) were treated in the same way except that the treatment was only given every third day instead of daily. The efficacy of the treatment was assessed by determining three target quantities in the pups: number of somatic T. canis larvae in samples of the liver, lung, kidney and gastrointestinal tract including content and musculature (euthanasia of the pups directly after birth), number of excreted eggs per gram of faeces and number of intestinal T. canis stages after washing out the intestine (euthanasia of the pups on the 35th day of life).

The percentage efficacy of emodepside in T1 was 99.9% in the case of the target quantity number of excreted eggs per gram of faeces, while it was 96.4% in the case of the target quantity number of somatic larvae in the digestive organs and 99.9% in the case of the target quantity number of intestinal stages in the intestine. In T2, an efficacy of 99.9% was also achieved for the target quantity number of excreted eggs per gram of faeces. The treatment scheme 2 achieved a percentage efficacy of emodepside of 93.8% in the case of the target quantity number of somatic larvae and a percentage efficacy of 98.7% in the case of the target quantity number of intestinal stages in the intestine. In both treatment schemes, the percentage efficacy of emodepside in the case of the individual target quantities, as well as the mean value obtained from these target quantities in each treatment group (T1=98.7%; T2=97.5%) were markedly above the least percentage efficacy of 90% which guideline 7 of the International Cooperation on Harmonisation of Technical Requirements for Registration of Veterinary Medicinal Products (VICH) demands for substances which have an anthelmintic effect. Both treatment schemes achieved a marked reduction in prenatal infection with T. canis in the pups.

Incompatibility phenomena were not observed either in the dam or in the pups.

1. Use of endoparasiticidal depsipeptides for producing pharmaceuticals for preventing vertical infection with endoparasites.

2. Use according to claim 1, wherein the endoparasiticidal depsipeptide employed is emodepside.

3. Use according to claim 1, for preventing vertical infection with endoparasites in dogs.
